(19) United States
(12) Patent Application Publication
(10) Pub. No.: US 2011/0275062 A1
(43) Pub. Date: Nov. 10, 2011

(54) Systems And Methods For Integrating A Single DNA Molecule Into A Molecular Electronic Device

(75) Inventors: Xuefeng Guo, New York, NY (US); Colin Nuckolls, New York, NY (US); James Hone, New York, NY (US); Alon Gorodetsky, New York, NY (US); Jacqueline K. Barton, San Marino, CA (US)

(73) Assignees: California Institute Of Technology, Pasadena, CA (US); The Trustees of Columbia University In the City of New York, New York, NY (US)

(21) Appl. No.: 12/955,310

(22) Filed: Nov. 29, 2010

Related U.S. Application Data
(63) Continuation of application No. PCT/US08/81147, filed on Oct. 24, 2008.
(60) Provisional application No. 61/057,683, filed on May 30, 2008.

(51) Int. Cl. C12Q 1/68; B05D 5/12; C23F 1/02; G01R 27/28; H01L 51/30; G01N 33/68; B05D 3/00; B82Y 99/00

(52) U.S. Cl. 435/6.1; 436/501; 427/122; 216/13; 324/649; 257/40; 977/742; 977/932; 977/750; 257/E51.038

(57) ABSTRACT

The disclosed subject matter provides a techniques for precisely and/or functionally cutting carbon nanotubes, e.g., single walled carbon nanotubes (“SWNTs”) and integrating a single nucleic acid molecule (e.g., a DNA molecule) into a gap formed into the carbon nanotubes. In one aspect, a method of fabricating a molecular electronic device includes disposing a SWNT on a base layer, forming a gap in the SWNT using a lithographic process, and disposing a single DNA strand across the gap so that each end of the nucleic acid contacts a gap termini. The disclosed subject matter also provides techniques for measuring the electrical properties (charge transport) of a DNA molecule which is integrated into an SWNT. Furthermore, a molecular electronic device including an SWNT with an integrated nucleic acid molecule is disclosed.

### CROSS-REFERENCE TO RELATED APPLICATIONS

This application claims priority to U.S. Provisional Application Ser. No. 61/057,683, filed May 30, 2008, the entirety of which is explicitly incorporated by reference herein.

### STATEMENT REGARDING FEDERALLY SPONSORED RESEARCH

This invention was made with government support under award numbers CHE-011752 and CHE-0641523 awarded by the Nanoscale Science and Engineering Initiative of the National Science Foundation; award number ECCS-0707748 awarded by the National Science Foundation's Nanoscale Interdisciplinary Research Teams (NIRT); career award number DMR-02-37860 awarded by the National Science Foundation; and grant number JKB-GM61077 awarded by the National Institutes of Health. The government has certain rights in the invention.

### BACKGROUND

1. Field

The disclosed subject matter relates to the field of molecular electronic devices.

2. Background Art

Since the elucidation of the double helical structure of DNA, scientists have been fascinated by the possibility that the stacked aromatic base pairs of DNA may enable charge transport (CT) over significant distances. The nature of the conductive properties of duplex DNA has consequently attracted substantial interest. Initial experiments featured photoinduced DNA-mediated CT between well-defined donor and acceptor sites.

Long-range CT can lead to oxidative damage in DNA over 200 A° away from the bound oxidant. DNA CT can also be sensitive to the integrity of the base-pair stack and to the coupling of the donors and acceptors with the DNA. Furthermore, DNA CT can be attenuated by a single base mismatch. Indeed, this sensitivity of DNA CT to the integrity of the base-pair duplex has prompted both the consideration of roles for DNA CT within the cell and the construction of electrochemical DNA-based sensors for mutations, base lesions and protein binding.

Numerous CT measurements on DNA strands bridging two electrodes have been carried out in an effort to establish the conductivity of DNA. These measurements have yielded a remarkably wide range of resistance values (1 to 1×10⁷ MΩ). For example, in some measurements, DNA ropes suspended on a metallic grid were found to behave as a semiconductor with a resistance on the order of 1 MΩ. However, other measurements demonstrated wide bandgap semiconducting behavior for DNA duplexes set between two nanoelectrodes using high-voltage electrostatic trapping, but later found insulating behavior for longer strands. In contrast, in other research, it was found possible to induce superconductivity at low temperatures in dehydrated DNA bundles on rhenium/carbon electrodes.

In general, the variability in the results obtained may be understood by considering the solution experiments, which show that DNA CT depends sensitively upon the integrity of the basepair stack, the absence of damage within the duplex, and the electrical connections to the duplex. It should be noted that measurements using both conducting atomic force microscopy (AFM) and scanning tunnelling microscopy (STM) under aqueous conditions have been carried out and show that well-matched DNA exhibits a low resistance (1-10 MΩ), as well as an increase in resistance with an intervening base mismatch. Also, STM measurements on DNA monolayers have shown effective charge transport for well-matched DNA oriented by the STM tip. However, none of these measurements was of a single duplex, but instead they were carried out for a collection of duplexes on the surface below the AFM or STM tip. Thus, in the conductivity measurements carried out so far, the integrity of the DNA was not well established, the connections to the duplex were not well defined, or the measurement was not definitively of a single DNA duplex.

### SUMMARY

Systems and methods are disclosed herein for integrating nucleic acid molecules, e.g., DNA, into molecular electronic devices. Further, systems and methods for detecting DNA-binding proteins and performing single nucleotide polymorphism (SNP) analysis are disclosed.

In an exemplary embodiment, a method of integrating a single DNA molecule into an electronic device is provided. The method includes disposing single walled nanotubes (SWNTs) on a base layer of the molecular electronic device, excising the SWNTs with oxygen ion plasma in order to form a gap in them, and bridging the gap with a single DNA molecule.

In some embodiments, the gap in the SWNTs is bridged by immersing the SWNTs in a buffer solution containing an amide solution, and reactivating the termini, or ends, of the gap with amine-modified DNA.

In some embodiments, one end of each of the two strands of the DNA are bound to the gap termini.

A single strand of the DNA can be bound between the ends of the gap termini.

The amide solution can have a pH of 7.2.

In some embodiments, the DNA is modified with only a single amine at the 5′ terminus.

The DNA can be prepared via solid phase synthesis on a controlled pore glass resin with an unprotected hydroxyl group at the 5′ terminus. The DNA can be modified with amines at the 3′ and 5′ termini.

In some embodiments, the oxidative etching of the SWNTs generates carboxylic acid functionalities on both sides of said gap.

In another exemplary embodiment, a method for measuring the conductivity of a DNA molecule is provided. The method includes disposing single walled nanotubes SWNTs on the base layer of a molecular electronic device, forming a gap in the SWNTs, bridging the gap with a single DNA molecule, and measuring the conductivity of the DNA molecule by applying a voltage across the gap.

In another exemplary embodiment, a molecular electronic device including an SWNT with an integrated nucleic acid molecule is disclosed.

### BRIEF DESCRIPTION OF THE DRAWINGS

FIGS. 1A-1C depict charts showing a method to cut and functionalize individual SWNTs with DNA strands according to some embodiments of the disclosed subject matter.

FIGS. 2A-2B depicts a chart and graph showing device characteristics for individual SWNTs connected with DNA according to some embodiments of the disclosed subject matter. More particularly, the graphs shown in FIGS. 2A-B depict the source-drain current versus V_{G} at a constant source-drain voltage (50 mV) before cutting (curve 1), after cutting (curve 2) and after connection with the DNA sequence shown (curve 3) for a semiconducting SWNT device (FIG. 2A) and a metallic SWNT device (FIG. 2B).

FIGS. 3A-3C depict charts and graphs demonstrating how mismatches in basepairs have a large effect on DNA conductance according to some embodiments of the disclosed subject matter.

FIGS. 4A-4B depict a chart and graph demonstrating how enzymes may be used to cleave the DNA between the ends of the SWNTs according to some embodiments of the disclosed subject matter. More particularly, FIG. 4B depicts the sour-drain current versus V_{G} at a source-drain voltage (50 mV) for a metallic SWNT device after cutting and reconnection with the DNA sequence of FIG. 4A before (curve 1) and after (curve 2) reaction with Alu I.

FIGS. 5A-5B depict charts showing the electrical characteristics of a device reconnected with well matched DNA at V_{sd}=−0.05 V according to some embodiments of the disclosed subject matter.

FIGS. 6A-B depict charts showing the electrical characteristics of a device reconnected with CA mismatched DNA at V_{sd}=−0.05 V (the sequence is: (CH2)₃-NHOCO-5′-AGT ACA GcC ATC GCG-3′; 3′-TCA TGT CaG TAG CGC-5′-OCOHN-(CH2)₃) according to some embodiments of the disclosed subject matter. 7A-B

FIGS. 7A-7B depict charts showing the electrical characteristics of a device rejoined with CA mismatched DNA at V_{sd}=−0.05 V according to some embodiments of the disclosed subject matter.

FIGS. 8A-8B depict charts showing the electrical characteristics of one rejoined device by GT mismatched DNA at V_{sd}=−0.05 V according to some embodiments of the disclosed subject matter.

FIGS. 9A-9B depict charts showing the electrical characteristics of a device reconnected with single-stranded DNA at V_{sd}=−0.05 V according to some embodiments of the disclosed subject matter.

FIG. 10 depicts a chart showing the results of various control experiments using partially cut devices that underwent the treatment of cutting, reconnection, dehybridization, and rehybridization according to some embodiments of the presently disclosed subject matter.

FIGS. 11A-11B depict charts showing the electrical characteristics of one device rejoined with well matched DNA missing the Alu I restriction site showing the sequence of cutting, reconnection and treatment by Alu I according to some embodiments of the disclosed subject matter.

Throughout the drawings, the same reference numerals and characters, unless otherwise stated, are used to denote like features, elements, components or portions of the illustrated embodiments. Moreover, while the disclosed subject matter will now be described in detail with reference to the Figures, it is done so in connection with the illustrative embodiments.

### DETAILED DESCRIPTION

The presently disclosed subject matter describes techniques for fabricating electronic devices with integrated nucleic acid molecules, e.g., DNA. It also provides techniques for obtaining measurements of the conductivity of a single DNA duplex when it is wired into a carbon electrode through covalent bonds. Systems and methods for performing single nucleotide polymorphism (SNP) analysis and detecting DNA-binding proteins are disclosed.

Techniques for measuring the conductivity of a single molecule covalently immobilized within a nanotube were previously disclosed in U.S. patent application Ser. Nos. 12/139,207, filed Jun. 13, 2008 and 12/139,218, filed Jun. 13, 2008, the entireties of which are explicitly incorporated by reference herein. According to those techniques, gaps are formed in SWNTs that may be reconnected by one or a few molecules attached to both sides of the gap through amide bond formation. The techniques allow molecules to be wired into metal electrodes by means of robust amide linkages. Moreover, the devices disclosed therein are sufficiently robust that aqueous environments can be used. Using those techniques, molecular devices can be made that are able to change their conductance as a function of pH, and others that are sensitive to the binding between protein and substrate, or that switch their conductance when the bridging molecules are photoswitched.

The presently disclosed subject matter describes systems and methods for integrating nucleic acid molecules, e.g., DNA, between carbon nanotube electrodes, e.g., SWNT electrodes. The presently disclosed subject matter also discloses measurements of the conductivity of a single DNA duplex when it is wired into a carbon nanotube electrode through covalent bonds.

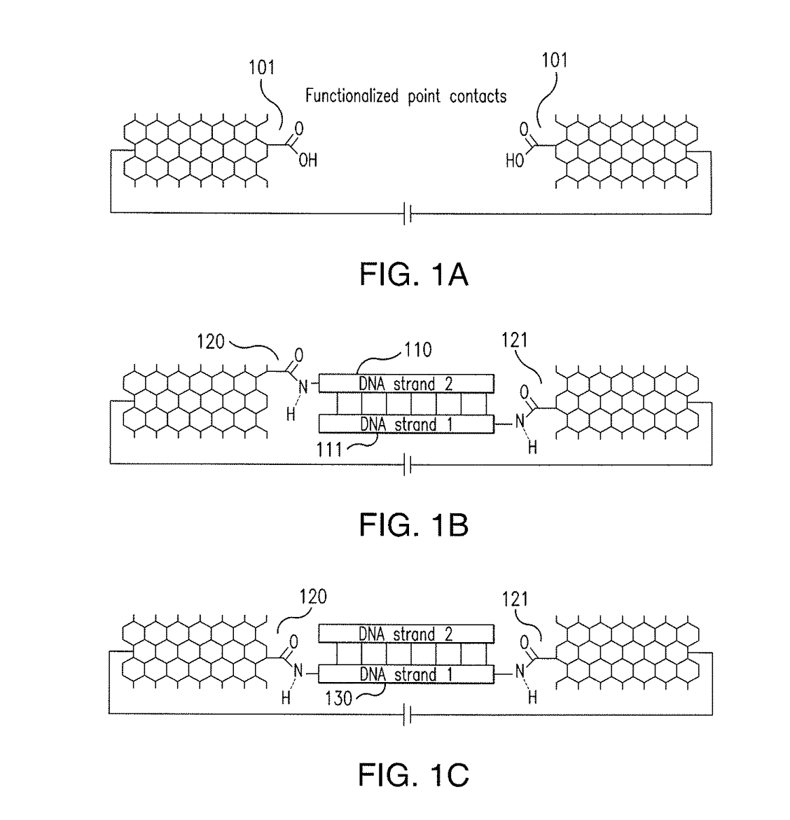

Fabrication of cut SWNT devices has been previously described in detail in U.S. patent application Ser. Nos. 12/139,207, filed Jun. 13, 2008 and 12/139,218, filed Jun. 13, 2008. SWNTs were grown using chemical vapor deposition (CVD) on highly doped silicon wafers with 300 nm of thermally grown silicon oxide on their surface. Metal electrodes having of 5 nm of Cr overlaid with 50 nm of Au were deposited through a shadow mask onto the carbon nanotubes. The silicon wafer can serve as a global back gate for the devices. A layer of polymethylmethacrylate (PMMA) can be spin-cast over the entire device structure. Ultra high-resolution electron beam lithography can be used to open a window in the PMMA. This process can expose a section of the SWNT only a few nanometers in length, which can be excised with an oxygen ion plasma. The oxidative etching of the carbon nanotube can generate carboxylic acid functionalities on both sides of the gap 101, as shown in FIG. 1A, which can be bridged with amine-terminated molecules.

The carbon nanotube gap can be reconnected with single nucleic acid molecules, e.g., DNA terminated with amines using a two-part process. First, freshly cut carbon nanotubes can be immersed in a buffer solution containing standard amide coupling and activating agents (Sulfo-NHS, EDCI). Then, the activated carbon nanotube termini can be reacted with amine-modified DNA to covalently bridge the gap with a single molecule. Given that the cross-sectional area of duplex DNA (˜3 nm²) is comparable to that of the SWNTs grown by the methods disclosed in the present subject matter, it is unlikely that more than one DNA duplex can fit lengthwise within the gap.

Two different methods can be explored to bridge these gaps. In one method, shown in FIG. 1B, one end each of the two strands of the DNA duplex 110, 111 are bound to the SWNT electrodes 120, 121. In a second method, shown in FIG. 1C, a single strand 130 is bound between the ends of the SWNT electrodes 120, 121. The method depicted in FIG. 1C allows for dehybridization/rehybridization with mismatched strands. Measurements for the presently disclosed subject matter were carried out under ambient conditions.

### Structures and Syntheses of DNA Molecules

Oligonucleotide Synthesis Unmodified oligonucleotides were prepared using standard phosphoramidite chemistry on an Applied Biosystems™ 394 DNA synthesizer, purified by high pressure liquid chromatography (HPLC) and characterized by mass spectrometry. Two strategies were used to synthesize DNA modified with only a single amine at the 5′ terminus and DNA modified with amines at both the 3′ and 5° termini. Oligonucleotides modified with an amine on the 5′ terminus only were prepared via solid phase synthesis on a controlled pore glass (CPG) resin with an unprotected hydroxyl group at the 5′ terminus. The 5′-OH was treated with a 120 mg/mL solution of carbonyldiimidazole in dioxane for two hours followed by an 80 mg/mL solution of 1,3-diaminopropane. The beads were thoroughly washed with dioxane, acetonitrile, and methanol leaving a free amine at the 5′ end. Oligonucleotides modified with amines on both the 3′ and 5′ termini were prepared via solid phase synthesis using reagents purchased from Glen Research™, Inc. The solid phase synthesis was performed on 3′-PT-Amino-Modifier C3 CPG with the 5′-Amino-Modifier C3-TFA phosphoramidite added in the final step of the solid phase synthesis to leave protected amines at both the 3′ and 5′ ends. The oligonucleotides were cleaved from the resin with concentrated ammonium hydroxide before being stringently purified by HPLC with a C18 column. The purified oligonucleotides were quantified via UV-Visible spectroscopy. Complementary single strand DNA was hybridized with its complement by heating equimolar amounts of each strand in buffer containing 5 mM phosphate, pH=7.1, 50 mM NaCl to 90° C., followed by cooling to ambient temperature.

Reaction Conditions for Reconnection and Subsequent Chemistries Reconnection Conditions:

Carboxylic acid activation: Newly-cut devices were incubated overnight in the BupH™ MES buffered Saline solution (pH 4.7, Pierce Biotech) containing 5 mM EDCI and 10 mM Sulfo-NHS. The devices were then removed from the solution, washed with fresh buffer solution, and dried with a stream of Nitrogen gas for device characterization.

Amide formation: The as-formed devices were incubated in the BupH™ Phosphate Buffered Saline solution (pH 7.2, Pierce Biotech) containing 10 uM duplex or single stranded DNA. The devices were then removed from the solution, washed with fresh buffer solution, and dried with a stream of Nitrogen gas for device characterization.

### Dehybridization/Hybridization Conditions:

The reconnected devices were immersed in a 50% formamide/DI water solution at 30° C. for one hour. Then the devices were removed from the solution, washed with DI water, and dried with a stream of Nitrogen gas. Subsequently, the above devices were incubated in BupH™ Phosphate Buffered Saline solution containing 10 uM of the corresponding single-stranded DNA. After one hour, the devices were removed from the solution, washed with fresh buffer, and dried with a stream of Nitrogen gas for device characterization.

### DNA Cutting Conditions:

The devices rejoined with duplex DNA were incubated in NEBuffer solution (pH 7.4, New England Biolabs Inc.) containing 100 units of the enzyme Alu I (New England Biolabs Inc.) at 37° C. for 6 hours. Then the devices were removed from the solution, washed with fresh buffer, and dried with a stream of N₂ gas for device characterization.

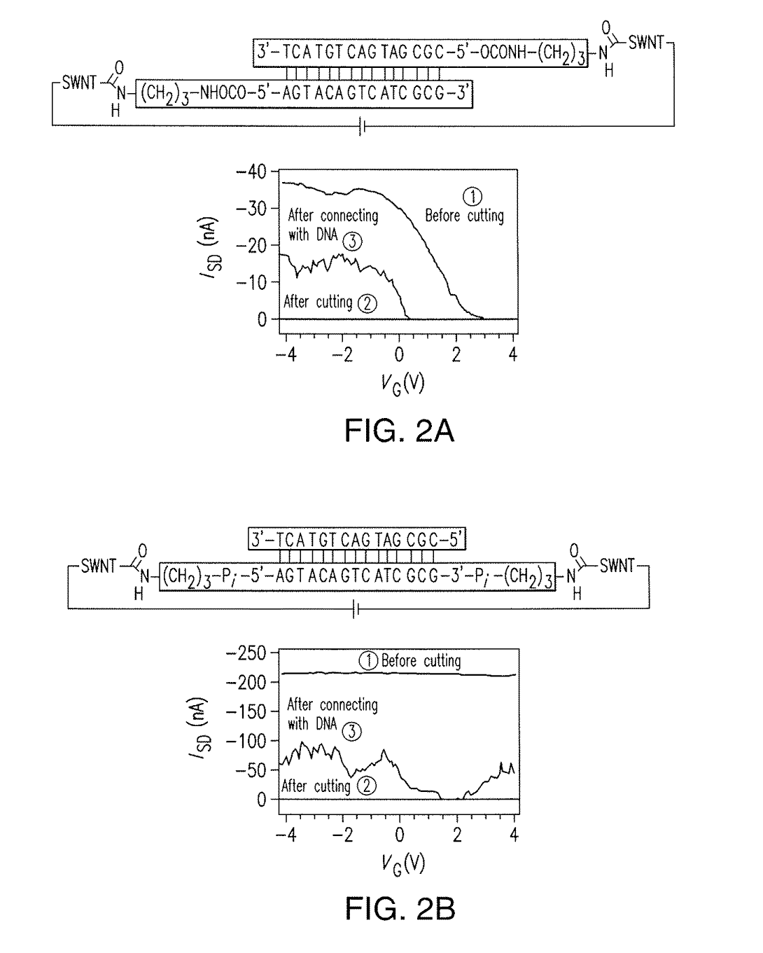

Referring now to FIGS. 2A and 2B, shown are two representative I-V graphs for the two different methods of DNA attachment disclosed in the present subject matter. No significant difference between the conductance measurements when using these two connection strategies was noted. In FIG. 2A, a DNA duplex functionalized on both strands with an amine at the 5′ end was utilized. In FIG. 2B, a DNA duplex containing a strand functionalized at both the 5′ and 3′ ends was used.

The curve labeled 1 on the I-V graph depicted in FIG. 2A shows the source-drain current (I_{SD}) as a function of the gate voltage (V_{G}) at a constant source-drain bias of 50 mV for the pristine nanotube. Before cutting of the SWNT, the device shown in FIG. 2A functions as a hole transporting semiconducting device, and the one shown in FIG. 2B functions as a metallic device. After cutting and initial treatment of the gap with coupling agents, the devices show no measurable current (as indicated by the curves labeled 2 in the I-V graphs of FIGS. 2A and 2B). The curves labeled 3 in the I-V graphs of FIGS. 2A and 2B illustrate the conductance of the two devices after reconnection with the two amine-modified DNAs.

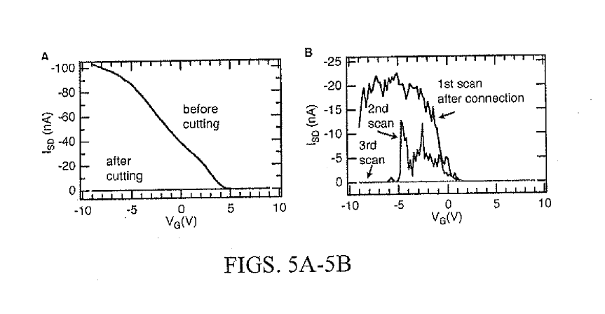

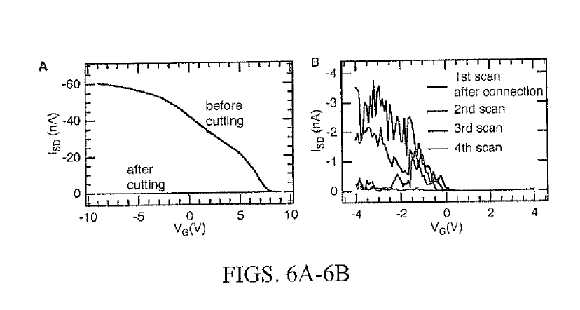

In the configurations depicted in FIGS. 2A and 2B, the reconnected carbon nanotube devices recover their original p-type semiconducting or metallic properties. However, the gate voltage that can be applied to the reconnected devices may be limited. For example, device breakdown sometimes occurs for gate voltages greater than 6V. Over time, at higher gate biases, the DNA bridges became poorer and poorer conductors until, ultimately, the current levels are at the noise level of the measurement (see, e.g., FIGS. 5 and 6). Table 1 summarizes the device characteristics measured in connection with the presently disclosed subject matter for various devices before cutting, after cutting and after reconnection with amine-terminated DNA sequences.

TABLE 1
Summary of the resistance values obtained before cutting and after reconnection

| Before Cutting | | | After reconnection | | |
|---|---|---|---|---|---|
| | Effective | | | Effective | DNA |
| Carbon nanotube | resistance* | | Type of | resistance* | conductance |
| type | (MΩ) | DNA sequence | linkage† | (MΩ) | (e²/h) |
| Semiconducting | 0.65 | Well matched | 5′ amine | 2.5 | 1.4 × 10⁻² |
| Semiconducting | 1.3 | Well matched | 5′ amine | 2.8 | 1.7 × 10⁻² |
| Semiconducting | 0.90 | CA mismatch | 5′ amine | 18 | 1.5 × 10⁻³ |
| Semiconducting | 0.48 | Well matched | 5′ & 3′ amine | 3.3 | 9.2 × 10⁻³ |
| Metallic | 0.23 | Well matched | 5′ & 3′ amine | 0.5 | 8.6 × 10⁻² |
| Metallic | 0.23 | CA mismatch | 5′ & 3′ amine | 155.0 | 1.7 × 10⁻⁴ |
| Metallic | 0.23 | GT mismatch | 5′ & 3′ amine | 111.0 | 2.3 × 10⁻⁴ |
| Metallic | 0.20 | CA mismatch | 5′ & 3′ amine | 67 | 3.9 × 10⁻⁴ |
| Semiconducting | 0.24 | GT mismatch | 5′ & 3′ amine | 31 | 8.5 × 10⁻⁴ |
| Metallic | 0.52 | Well matched | 5′ & 3′ amine | 36 | 7.3 × 10⁻⁴ |
| | | (Alu I) | | | |
| Semiconducting | 1.5 | Single-stranded | 5′ & 3′ amine | 3.0 | 1.7 × 10⁻² |

*Resistance values were calculated using a gate bias of −4 V and a source-drain bias of −50 mV
†The 5′ amine linkage correspond to a —OCONH—(CH)₃—NH₃ linker on the 5′ ends of both strands. The 3′ and 5′ amine linkage corresponds to a —Pi—(CH₂)₃—NH₂ linker on both the 3′ and 5′ ends of one strand.

By using the method disclosed in the present subject matter, a total of 10 working electronic devices were obtained out of 370 that were tested. FIGS. 5A-11B depict experimental details of the electrical measurements carried out on these devices.

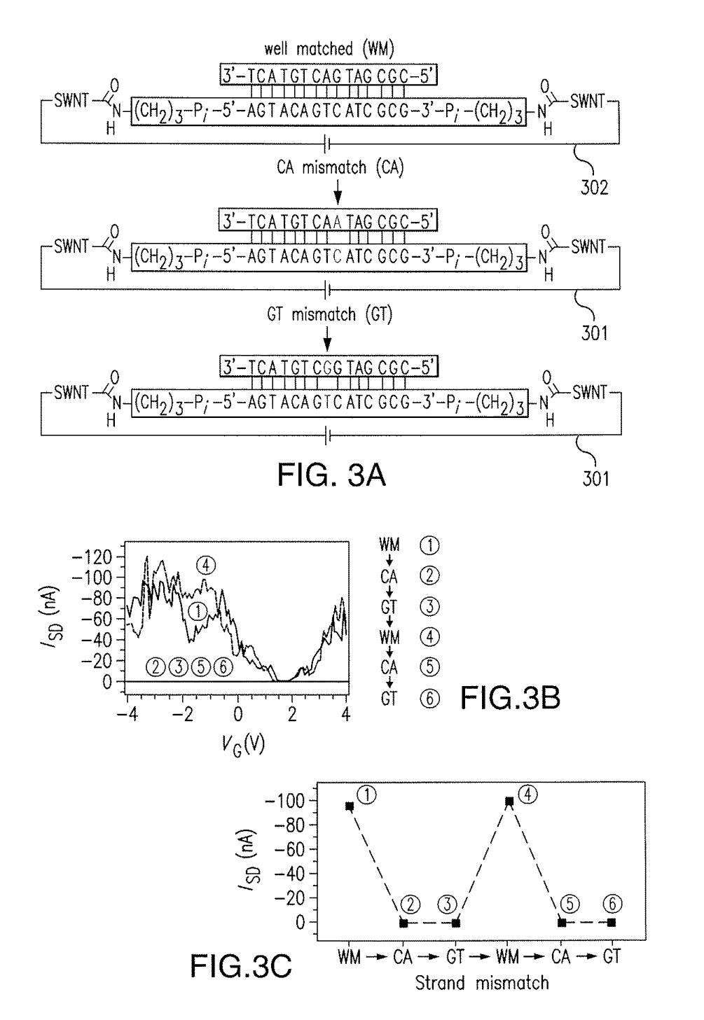

Devices were also reconnected with mismatched DNA, as it has been shown in a variety of experiments that single-base mismatches dramatically attenuate CT. The DNA duplexes and mismatches explored in the presently disclosed subject matter are depicted in FIG. 3A. The mismatched devices 301 were found to have higher resistance than corresponding devices reconnected with well-matched DNA 302. These results could not be compared quantitatively with those on the well-matched duplex, because different devices were fabricated to test the different duplexes. A device was therefore first reconnected with well matched DNA duplexes functionalized with the amines on the 5′ and 3′ termini of one strand, and then the duplex was dehybridized using a 1:1 solution of formamide and deionized water at 30° C. and rehybridized with different complements (FIGS. 3A-3C).

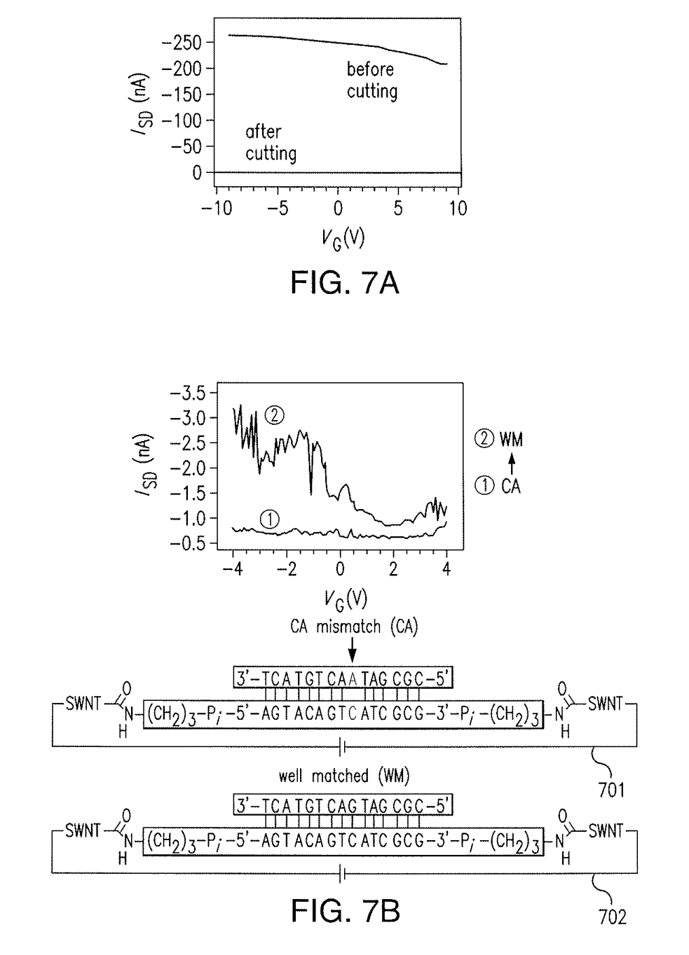

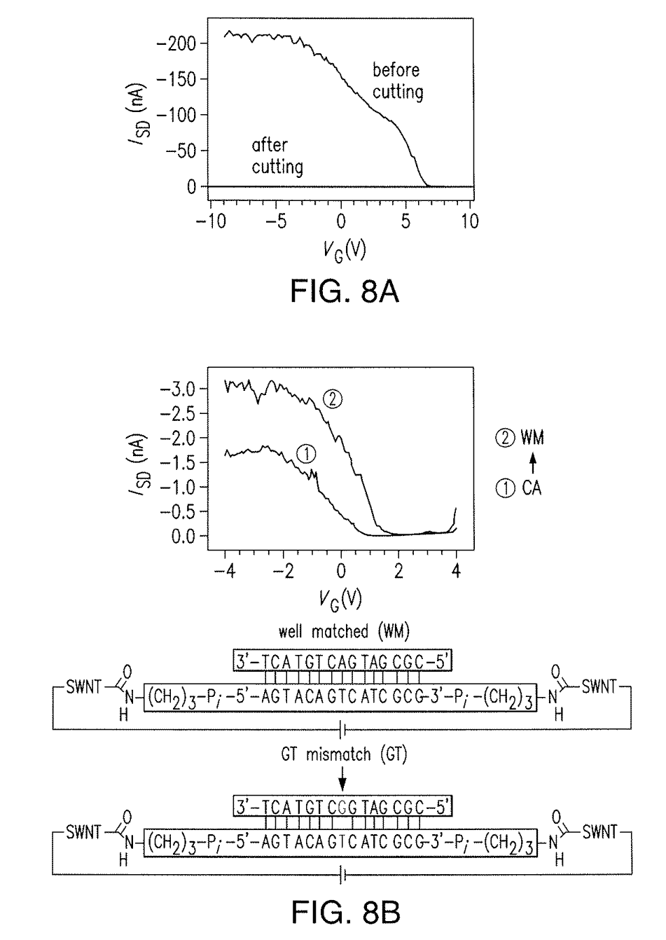

FIG. 3B depicts the corresponding current-voltage curves for the different rehybridization sequences shown in FIG. 3A. FIG. 3C depicts the current at V_{G}=−3 V curves for the different rehybridization sequences shown in FIG. 3A at a constant source drain bias of 50 mV. Rehybridization with the complement so as to generate a CA mismatch reduced the current significantly and yielded an increase in the on-state resistance of nearly 300-fold from 0.5 MΩ to 155 MΩ (FIG. 3C). Replacing the complement featuring a CA mismatch with a complement featuring a GT mismatch yielded no changes in device characteristics. However, the original on-state resistance and nanoamp current levels could then be recovered by replacing the GT mismatched complement with the original well-matched sequence. Importantly, the device could be taken through multiple dehybridization/rehybridization cycles, as shown in FIGS. 3B and 3C. As further confirmation that CT in the carbon nanotube gap is DNA-mediated, reconnected separate devices were reconnected first with DNA featuring a GT mismatch or DNA featuring a CA mismatch 701 as shown in FIGS. 7A-8B. Dehybridization of the mismatched DNA and replacement with well-matched DNA yielded an increase in the current and a decrease in the on-state resistance in both instances. It is important to note that the thermodynamically stable GT mismatch produced an effect that is identical to that found with the thermodynamically destabilizing CA mismatch. As has been found in solution experiments, the attenuation in DNA CT seen with mismatches does not correlate with thermodynamic stability of the duplex. Ultrafast spectroscopic experiments indicate that DNA CT depends upon the sequence-dependent dynamics of DNA. Certainly, the changes observed in the presently disclosed subject matter in the electrical characteristics of the device with mismatches cannot be due to poorer stability of the DNA.

Although the mismatch experiments provide strong evidence that the observed signals do not result from ionic conduction from the DNA molecules, as an additional control, newly cut devices were subjected to the same reconnection conditions but with the DNA excluded. After removal from the solution and rinsing, all of the devices treated in this manner remained at open circuit with no measurable current.

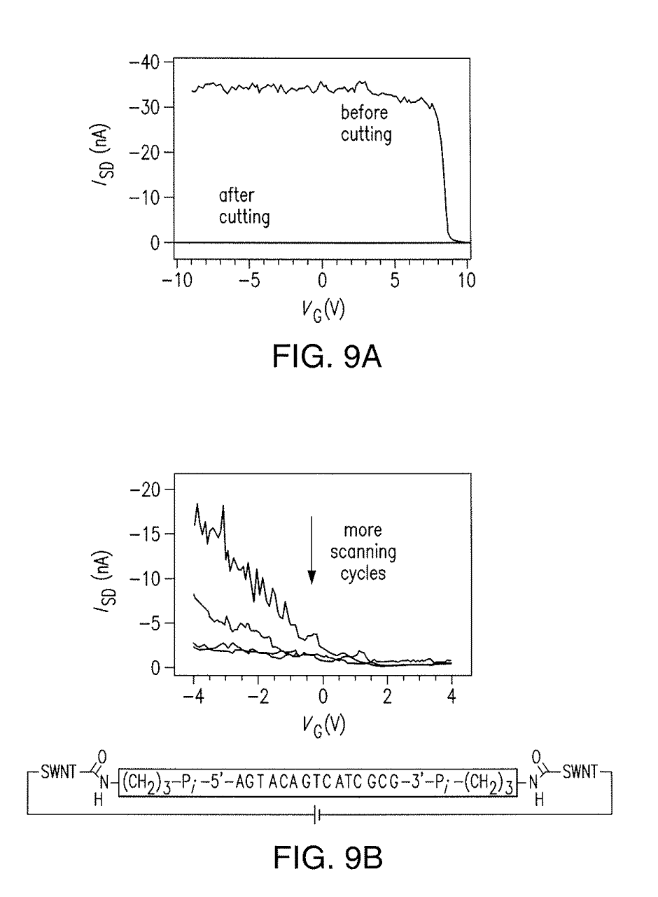

Devices were also reconnected with single-stranded DNA featuring amines at both the 5′ and 3′ ends but without its complement. Although carbon nanotube gaps could be bridged with the single-stranded DNA, the resulting devices were found to be highly unstable (e.g., as shown in FIGS. 9A-9B). After three voltage cycles, the current passing through single stranded DNA degraded to open-circuit levels. Such instability may result from voltage-induced oxidation of the exposed nucleobases and was not observed with duplex DNA.

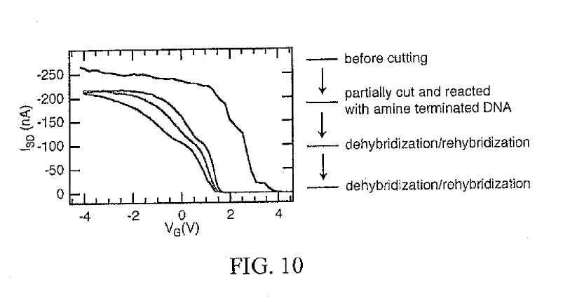

Additional control experiments were performed to determine if non-specific absorption of DNA could be responsible for the conduction changes during dehybridization/rehybridization. Devices were partially cut with a shorter oxygen plasma treatment before being taken through the sequential steps of reconnection and exchanges from matched to mismatched sequences. In essence, the SWNT is only nicked, not cut completely through, so the electrical connection is maintained. The devices treated in this way displayed little change in either the resistance or threshold voltage as shown in FIG. 10.

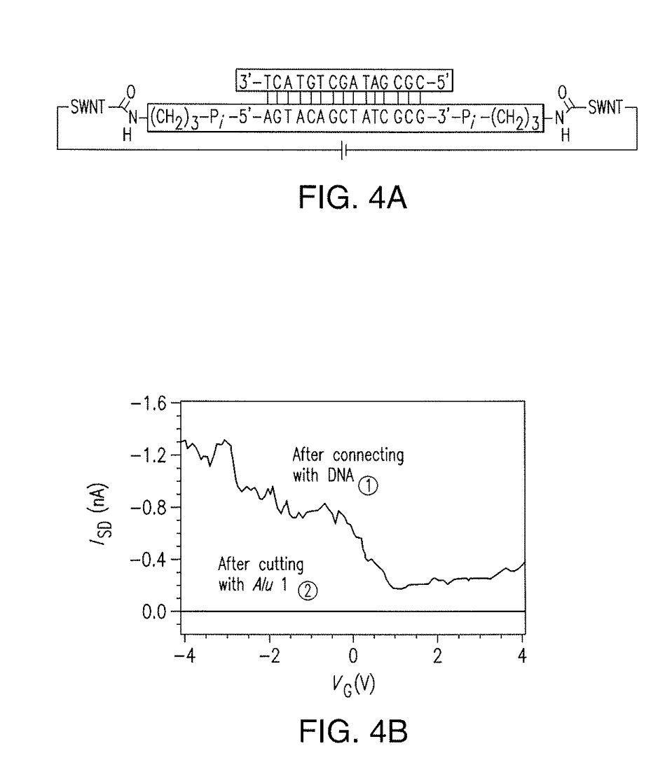

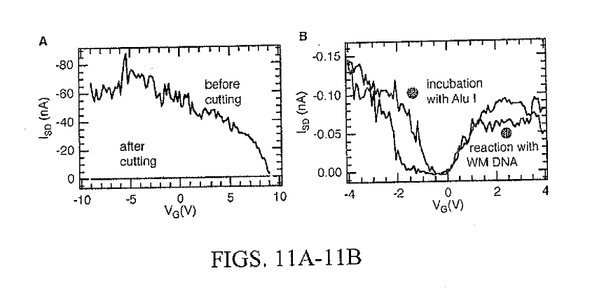

As a final test that the duplex DNA within the gap adopts a native conformation under the conditions of the experiment, Alu I, a blunt end restriction enzyme, was used to cut the DNA (FIG. 4). Alu I only cuts DNA that is in its native conformation. Devices were reconnected with duplex DNA containing the restriction sequence 5′-AGCT-3′. The device was subsequently incubated with Alu I, resulting in a concomitant decrease in the current to the noise limits of the measurement. As another control, a device reconnected with a nearly identical sequence that featured the sequence 5′-AGTC-3′ in place of the restriction site was incubated with Alu I. In this instance, no significant change was observed in the electrical characteristics of the device as shown in FIGS. 11A-11B. These data support the observation of a sequence-specific restriction event. The enzyme is able to cleave its target sequence, yielding no detectable current in the device. Under the experimental conditions presented, then, the DNA duplex is intact, and the results suggest that it adopts a native conformation. Further, because Alu I was able to cut the DNA, proteins that bind DNA can be recognized by the method.

By using the aforementioned data, experimental values found here may now be placed in the proper context to establish a range for the conductivity of a single, intact DNA duplex, The measurements conducted in the presently disclosed subject matter place the resistance of well-matched DNA duplexes with ˜6 nm length in the range of 0.1-5 MΩ as shown in Table 1. For comparison, based on the bulk c-axis resistance, highly oriented pyrolytic graphite (HOPG) with similar dimensions should also have a resistance of ˜1 MΩ. This value may be estimated by substituting a HOPG stack of equivalent diameter for the double-stranded DNA. Thus, it appears that DNA, in its well-matched and well-stacked duplex form, behaves electrically much like an array of stacked aromatic graphite planes. Importantly, just as seen in solution, the presence of intervening mismatches attenuates DNA-mediated CT. This attenuation leads to a ˜300-fold increase in resistance. Such an increase in the resistance of mismatched DNA is consistent with previously reported STM measurements. Also, it should be noted that even within the measurements provided in the presently disclosed subject matter, the covalent s-bonded linkages at the termini of the DNA duplex must also decrease the conductivity observed versus the conductivity expected with coupling directly into the base-pair stack. Therefore, the values obtained represent the upper limits of the resistance of the DNA p-stack.

In conclusion, a method for integrating a single DNA duplex within an electrical device has been outlined. The DNA molecules are covalently wired into electrical circuits through robust amide linkages that are stable over a wide range of chemistries and conditions. The experiments described in the presently disclosed subject matter illustrate the ability of DNA to mediate CT over significant distances and allow for the direct measurement of the resistance of a single well-matched DNA molecule. DNA, if in its native conformation, and containing a stack of aromatic heterocycles in its core, resembles the aromatic stacked planes of graphite with respect to electrical characteristics.

The foregoing merely illustrates the principles of the disclosed subject matter. Various modifications and alterations to the described embodiments will be apparent to those skilled in the art in view of the teachings herein, It will thus be appreciated that those skilled in the art will be able to devise numerous techniques which, although not explicitly described herein, embody the principles of the disclosed subject matter and are thus within the spirit and scope of the disclosed subject matter.

We claim:

1. A method for integrating a single nucleic acid molecule into a molecular electronic device comprising:
(a) disposing a carbon nanotube on a base layer of said molecular electronic device;
(b) forming a gap in said carbon nanotube; and
(c) bridging said gap with a single nucleic acid molecule.

2. The method of claim 1, wherein said nucleic acid comprises DNA.

3. The method of claim 1, wherein said carbon nanotube comprises a single walled carbon nanotube (SWNT).

4. The method of claim 1, wherein said gap is formed by oxidatively etching said carbon nanotube with an oxygen ion plasma.

5. The method of claim 1, wherein said bridging comprises:
(a) immersing said carbon nanotube in a buffer solution containing amide coupling and activating agents to form an amine modified nucleic acid molecule; and
(b) reacting the termini of said gap with said amine modified nucleic acid molecule.

6. The method of claim 5, wherein the amine modified DNA comprises a duplex DNA molecule.

7. The method of claim 6, wherein one end of each of the two strands of the duplex DNA molecule are bound to the termini of said gap.

8. The method of claim 6, wherein a single strand of the duplex DNA molecule is bound to the termini of said gap.

9. The method of claim 5, wherein the buffer solution has a pH of 7.2.

10. The method of claim 2, wherein the DNA is modified with a single amine at the 5′ terminus.

11. The method of claim 7, wherein the DNA is prepared via solid phase synthesis on a controlled pore glass resin with an unprotected hydroxyl group at the 5′ terminus.

12. The method of claim 2, wherein the DNA is modified with amines at the 3′ and 5′ termini.

13. The method of claim 4, wherein the oxidative etching of said carbon nanotube generates carboxylic acid functionalities on both sides of said gap.

14. A method for measuring the conductivity of a nucleic acid molecule comprising:
(a) disposing a carbon nanotube on the base layer of a molecular electronic device;
(b) forming a gap in said carbon nanotube;
(c) bridging said gap with a nucleic acid molecule; and
(d) measuring the conductivity of said nucleic acid molecule by applying a voltage across said gap.

15. A molecular electronic device comprising:
(a) a base layer having first and second sides;
(b) a carbon nanotube cut into a first portion and a second portion, being disposed on the same side of the base layer such that a gap is formed between the first portion and the second portion of the cut carbon nanotube;
(c) a single nucleic acid molecule positioned in the cut carbon nanotube gap and bridging the first portion to the second portion of the cut carbon nanotube.

16. The device of claim 15, wherein said nucleic acid molecule comprises DNA.

17. The device of claim 16, wherein said carbon nanotube comprises a single walled carbon nanotube (SWNT).

18. The device of claim 15, wherein the carbon nanotube is cut by oxidatively etching said carbon nanotube with an oxygen ion plasma.

19. The device of claim 16, wherein, said DNA is amine modified.

20. The device of claim 19, wherein the amine modified DNA comprises a duplex DNA molecule.

21. The device of claim 20, wherein one end of each of the two strands of the duplex DNA molecule are bound to the termini of the carbon nanotube gap.

22. The device of claim 20, wherein a single strand of the duplex DNA molecule is bound to the termini of the carbon nanotube gap.

23. The device of claim 16, wherein the DNA is modified with a single amine at the 5′ terminus.

24. The device of claim 16, wherein the DNA is prepared via solid phase synthesis on a controlled pore glass resin with an unprotected hydroxyl group at the 5′ terminus.

25. The device of claim 16, wherein the DNA is modified with amines at the 3′ and 5′ termini.

26. The device of claim 18, wherein the oxidative etching of said carbon nanotube generates carboxylic acid functionalities on both sides of the carbon nanotube gap.

27. A method of detecting a nucleic acid binding protein comprising:
(a) disposing a carbon nanotube on the base layer of a molecular electronic device;
(b) forming a gap in said carbon nanotube;
(c) bridging said gap with a nucleic acid molecule;
(d) incubating the device with a nucleic acid binding protein;
(e) measuring the conductivity of said nucleic acid molecule by applying a voltage across said gap.

28. The method of claim 27, wherein the nucleic acid binding protein consists of Alu I.


SEQUENCE LISTING (7 sequences)

SEQ ID NO: 1 - DNA, 15 bp, Artificial Sequence
  Description of Artificial Sequence Synthetic oligonucleotide
agtacagccatcgcg

SEQ ID NO: 2 - DNA, 15 bp, Artificial Sequence
  Description of Artificial Sequence Synthetic oligonucleotide
cgcgatgactgtact

SEQ ID NO: 3 - DNA, 15 bp, Artificial Sequence
  Description of Artificial Sequence Synthetic oligonucleotide
agtacagtcatcgcg

SEQ ID NO: 4 - DNA, 15 bp, Artificial Sequence
  Description of Artificial Sequence Synthetic oligonucleotide
cgcgataactgtact

SEQ ID NO: 5 - DNA, 15 bp, Artificial Sequence
  Description of Artificial Sequence Synthetic oligonucleotide
cgcgatggctgtact

SEQ ID NO: 6 - DNA, 15 bp, Artificial Sequence
  Description of Artificial Sequence Synthetic oligonucleotide
cgcgatagctgtact

SEQ ID NO: 7 - DNA, 15 bp, Artificial Sequence
  Description of Artificial Sequence Synthetic oligonucleotide
agtacagctatcgcg